(19) United States
(12) Patent Application Publication
(10) Pub. No.: US 2020/0146718 A1
(43) Pub. Date: May 14, 2020

(54) NEUROVASCULAR PUNCTURE-AVOIDANT SHEATH

(71) Applicant: Kris V. Tamirisa, La Jolla, CA (US)

(72) Inventor: Kris V. Tamirisa, La Jolla, CA (US)

(21) Appl. No.: 16/741,343

(22) Filed: Jan. 13, 2020

Related U.S. Application Data
(63) Continuation-in-part of application No. 15/170,113, filed on Jun. 1, 2016, now Pat. No. 10,532,186.

(51) Int. Cl. A61B 17/34; A61M 25/01

(52) U.S. Cl. CPC A61B 17/3496; A61B 17/3403; A61M 25/0108; A61B 2017/3413; A61B 17/3401

(57) ABSTRACT

A device and method for a guidance sheath is provided which is configured to prevent laceration or puncture of nerves, blood vessels and surrounding tissue, during positioning of a coaxially located needle employed for various medical procedures, such as, the administration of an anesthetic blockade, neuromonitoring, electromyography or a therapeutic intervention. The body of the sheath may be electrically conductive to allow communication of electric current to exposed portions of an exterior surface of the body of the device which are not coated with an insulation.

This application is a Continuation in Part application to U.S. patent application Ser. No. 15/170,113, filed on Jun. 1, 2016, which is incorporated herein in its entirety by this reference thereto.

### BACKGROUND OF THE INVENTION

### 1. Field of the Invention

The present invention relates to a device and method employed during the provision of regional anesthesia. More particularly the disclosed device and method relate to a guidance sheath which is configured to prevent laceration or puncture of nerves, blood vessels and surrounding tissue, during positioning of a coaxially located needle employed for various medical procedures, such as, the administration of an anesthetic blockade, neuromonitoring, electromyography or a therapeutic intervention. Further the unique tip renders the device more easily discerned for position and angle when viewed on a display screen using electronic medical imaging.

### 2. Prior Art

In the practice of local or regional anesthesia, a physician administers anesthesia to a specific body region. Such is regularly practiced during surgery, or for relief of pain subsequent to surgery, or as a means for extended pain relief due to trauma or chronic pain caused by disease. In many surgical cases, a local or regional anesthesia is preferable to general anesthesia because of increased safety. Further, the ability of a local blockade continues after such surgery as a means to control postoperative pain control.

During the administration of local or regional anesthesia, the physician delivers a local anesthetic in close proximity to a target or nerve plexus. When precisely placed, a neural blockade is established to eliminate or significantly reduce sensed pain by the patient in the area of the delivered local aesthetic.

Electrical nerve stimulation in the past has been a commonly used method for localizing or discerning target nerves, prior to the injection of local anesthetic. Such electrical nerve stimulation during local or regional anesthesia employs electrical stimulus delivered by a needle to obtain a defined response (muscle twitch or sensation). In this fashion, the physician is able to observe and locate a peripheral nerve or nerve plexus desired with electric pulses communicated by a needle which cause localized patient movements.

In more recent years, ultrasound has been employed by physicians during needle placement. In such procedures an ultrasound generating device generates viewable depictions of the interior of the body of the patient, and concurrently shows the needle or probe device and its location. Some physicians may use one means, for determining nerve location, or the other and in many case physicians will uses both means for determining if the distal end of the anesthetic delivery needle is properly placed to anesthetize the desired nerve or body location for the procedure.

Once the nerve or locale for injection has been determined, relative to the needle, a determined amount of a local anesthetic is communicated to a delivery point in close proximity to the determined nerve site to block nerve conduction and thereby provide a sensory and motor block for surgery and/or, eventually, analgesia for pain management.

Such needle delivered electrical nerve stimulation can be used for a single-injection technique, as well as to provide guidance during a subsequent insertion of, for example, a continuous nerve block catheter.

However, successful provision of regional anesthesia is particularly dependant upon the accurate placement of the needle providing electrical impulses, as well as the subsequent positioning of the needle communicating the anesthetic, proximate to the target nerve or nerves. Further, whether it be the delivery of anaesthesia drugs to the determined site, or the communication of electrical stimulation prior to such drug delivery, needles employed for both such instances must be accurately moved through body tissue and placed both to determine and to anesthesize the plexus or target site.

As a consequence of the need to move a pointed object through body tissue during such procedures, the potential for injury to adjacent nerves, blood vessels, and body tissue from the sharp point of the needle delivering the electrical stimulus, as well as the drug delivery needle or catheter, continues to be a significant risk factor to the patient.

Probing and drug delivery procedures using elongated needles or similar instruments have the potential to cause significant harm to the patient. Such can easily occur from unintended punctures and lacerations to surrounding nerves and blood vessels. While the risk of such injury may reduce in relation to the skill of the physician, such is not eliminated since the physician is using two dimensional sound generated images, and reflex reactions, and cannot actually view potential delivery site for the needle point, which is being translated within the body of the patient. While probes with coverings have been employed by medical professionals to help reduce the potential for harm, conventional needle sheaths themselves have been known themselves, to cause internal injury to delicate nerves and blood vessels adjacent an intended anesthetic delivery site.

The device herein is adapted to surround a needle employed for medical procedures such as, for example, a procedure for delivery of local anaesthesia. So positioned, it is adapted to reduce the chance of puncture or laceration of nerves and blood vessels and body tissues during the positioning of a sharp needle point at a determined location.

### SUMMARY OF THE INVENTION

The device herein disclosed and described provides an elongated protective sheath which is adapted to engage upon and surround a needle while being advanced into the body tissues of a patient. The elongated sheath has an axial cavity communicating between a first opening providing a lumen adapted for an engagement with a needle base having a needle extending into the axial cavity.

The sheath has a second opening adapted for translation of the distal end of the needle located in the lumen defined by the axial passage, to project from the second opening when triggered. A drug delivery needle in an engaged configuration within the axial cavity of the surrounding sheath is thus shielded from contact with nerves, blood vessels, and the tissue of the patient during positioning of the sheath and coaxially positioned needle at a blockade site. Further, the sheath may also be employed upon a nerve block needle used for nerve stimulation to prevent tissue and nerve damage during locating the needle to communicate an electric charge to a nerve.

Particularly preferred, in all modes of the device herein, is the formation of the distal end of the sheath, in a bulbous shape, relative to the linear cylindrical shape of the sheath extending from the first end to the bulbous distal end. The bulbous shaped portion of the sheath, at the distal end and adjacent an annular recess depending into the circumference of the exterior surface of the body of the sheath, is preferably sized with a diameter at a smallest, which adapted to allow passage of the needle axially through the second opening located at the distal end.

The largest diameter of the spherical portion is to equal or slightly less than the diameter of the cylindrical portion of the body of the sheath running from the annular recess toward the first end of the sheath. Currently a diameter of the spherical portion having an diameter to yield a circumference of the spherical portion between fifty to ninety five percent of the circumference of the first or cylindrical portion of the body of the sheath is preferred for a number of reasons noted herein.

The bulbous portion at the distal end of the sheath positions a highly curved distal end to communicate with the tissues of the patient during advancement of the needle covered by the sheath therethrough. This is unlike the pointed or straight cylindrical walled sheaths or covers conventionally in use. The highly curved area of the spherical portion, extending radially from the second opening, back to the annular recess, positions curved surfaces facing all tissue contacting the distal end of the sheath during use. No matter the direction of movement of the distal end of the sheath, the patient tissue will contact a curved surface.

Conventional sheaths or shrouds lack such a smooth curved contact surface radially extending from the second opening. Instead, such conventional devices present a forward facing sidewall or the like which can itself cut or lacerate patient tissue during advancement and positioning in the patient.

An additional advantage found with the bulbous portion adjacent an annular recess during ultrasonography assisted guided positioning, or fluoroscopically guided positioning, or computed tomography guided positioning, is a more easily discerned location of the distal end of the needle-surrounding sheath, within the body of the patient. The spherical portion or bulbous shape at the distal end of the sheath, adjacent the annular recess, provides the physician with certainty as to the location of the distal end of the sheath with all such sound and RF-aided positioning, which create video depictions of the body interior.

This spherical portion or bulbous shape is viewable from a wide variety of angles by the physician with certainty as to the location of the distal end of the sheath relative to surrounding tissues. This is unlike a conventional cylindrical shaped sheaths or covers which employ a linear sidewall surrounding an opening at the second end which is hard to ascertain in angled 2D views which occur frequently in the two dimension video depicted viewing noted above. Consequently, it has been found the physician can more easily determine the angular portioning of the axis of the sheath and needle, with the bulbous or substantially spherical portion and recess at the distal end since with practice the angular position can be discerned by viewing the tissue through the annular recess direction of the bulbous portion during movement.

The sheath disclosed herein, is adapted to connect in an engaged position, to surround any needle or elongated device, being advanced into a patient. In this engaged position the sheath protects the nerves, blood vessels and tissues of a patient from the needle, and helps eliminate lacerations or punctures caused by the points and exposed edges of the cylindrical openings of conventional needle-surrounding sheaths and the like. Further, so engaged coaxially with a needle, the device provides enhanced viewing on electronically generated images and easy visual determination of the distal end of the sheath or sheath at a wide variety of angular views, because the novel spherical portion adjacent the annular recess, is easily discernable on video screens of depicting images generated by sonic, RF, or computer-aided positioning devices. Further, the sheath may be surfaced with angular indentations rendering it more visible during an ultrasound guided procedure, or a polymeric coating having hollow nano microspheres encapsulated in the cured material, such as microspheres from Cospheric Innovations of Santa Barbara, Calif.

The sheath device herein can be adapted to engage and surround a wide variety of needles, or other elongated delivery devices which must be positioned within the body of a patient with a high degree of precision such as noted above. So engaged the device concurrently provides the protection from puncture and laceration, as well as improved viewing using electronic and sonic generated images. It should be noted that the sheath device herein is employable with both human and animal patients. Consequently, the description herein focusing upon needles employed for positioning at a target or nerve plexus in humans, should not be considered limiting in scope.

With respect to the above description, before explaining at least one preferred embodiment of the herein disclosed needle shielding and positioning sheath invention in detail, it is to be understood that the invention is not limited in its application to the details of construction and to the arrangement of the components in the following description or as illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based, may readily be utilized as a basis for designing of needle surrounding structures, methods and systems for carrying out the several purposes of the present disclosed device. It is important, therefore, that the claims be regarded as including such equivalent construction and methodology insofar as they do not depart from the spirit and scope of the present invention.

It is an object of the invention to provide a needle surrounding sheath, which adapted for an engagement to surround needles being positioned in a patient.

It is another object of the invention to provide such a sheath, which has a substantially spherical or bulbous portion positioned at the distal end, radially surrounding a second opening for translation of a coaxial positioned needle therethrough.

It is a further object of the invention to provide a such a needle surrounding sheath, in which the spherical portion or bulbous portion at a second end and adjacent an annular recess, provides enhanced viewing and distal end position determination on electronically generated views thereof.

Further objects of the invention will be brought out in the following part of the specification, wherein detailed description is provided for the purpose of fully disclosing the invention but without placing limitations thereon.

### BRIEF DESCRIPTION OF DRAWING FIGURES

FIG. 1 depicts the device herein in an engaged position with the sheath surrounding a needle positioned within an axial passage therein, and a substantially spherical portion positioned at distal end, adjacent an annular recess.

FIG. 2 shows a sectional view of the device similar to that of FIG. 1, having a first end adapted for removable or other operative engagement with a body engaged at a first end of a needle extending into the axial passage of the body forming the sheath.

FIG. 3 depicts the device herein in a similar fashion to that of FIG. 2, and showing the first end in another configuration adapted for operative engagement with the body engaged to a first end of the needle.

FIG. 4 shows the device in a similar fashion to that of FIG. 1, and depicts the second end having the substantially spherical portion adjacent an annular recess, which is encircled by dotted line referring to the enlarged shapes of FIGS. 4a and 4B and 4c.

FIG. 4A shows a substantially spherical portion with an elongated globe-like or substantially circular shape having a curved surface radially communicating around and away from the second opening, toward the annular recess which is positioned in-between a first linear portion of the body of the sheath and the spherical portion. Also shown are the three diameters D1, D2, and D3.

FIG. 4B shows the substantially spherical portion similar to that of FIG. 4A, but with more elongated sides to form a more substantially spherical shape, and which positions a curved wall surface extending radially around the second opening to the annular recess.

FIG. 4C depicts the substantially spherical portion similar to that of FIGS. 4A and 4B, but with a sidewall having a less pronounced curved surface, and which positions a curved end surface extending radially from the second opening toward the annular recess.

FIG. 5 depicts the exterior surface of the sheath at and adjacent the distal end, configured with a surfacing of the exterior surface adapted to enhance a sonic, RF, or computer aided electronic video display depiction thereof.

FIG. 6, depicts the exterior surface of the sheath at a first portion running toward the first end from the annular recess, configured with a surfacing adapted configured to enhance a sonic, RF, or computer generated video display depiction thereof.

FIG. 7 shows a mode of the device having a sheath which is electrically conductive and has an insulating coating covering all the exterior surface but for a portion adjacent to or encircling the second opening from which the distal end of the needle may project.

FIG. 8 shows the device as in FIG. 7 with the insulated exterior surface extending to the area surrounding the second opening and showing in dotted line the tip which is depicted in enlarged modes in FIGS. 8A-8C.

FIG. 8A shows the tip of the device herein enlarged to show the conductive portion of the exterior surface, adjacent to or surrounding the second hole, and showing reflective surfacing adjacent to the conductive portion, thereby providing the surgeon two points of positional reference adjacent each other for better position determination.

FIG. 8B is shown configured with the conductive portion adjacent the reflective portion and having an extended tip.

FIG. 8C shows the tip of the device having an extra elongated tip portion adjacent and extending forward from the recess and having the reflective portion immediately adjacent the conductive portion on the exterior of the device.

### DETAILED DESCRIPTION OF THE PREFERRED EMBODIMENTS OF THE INVENTION

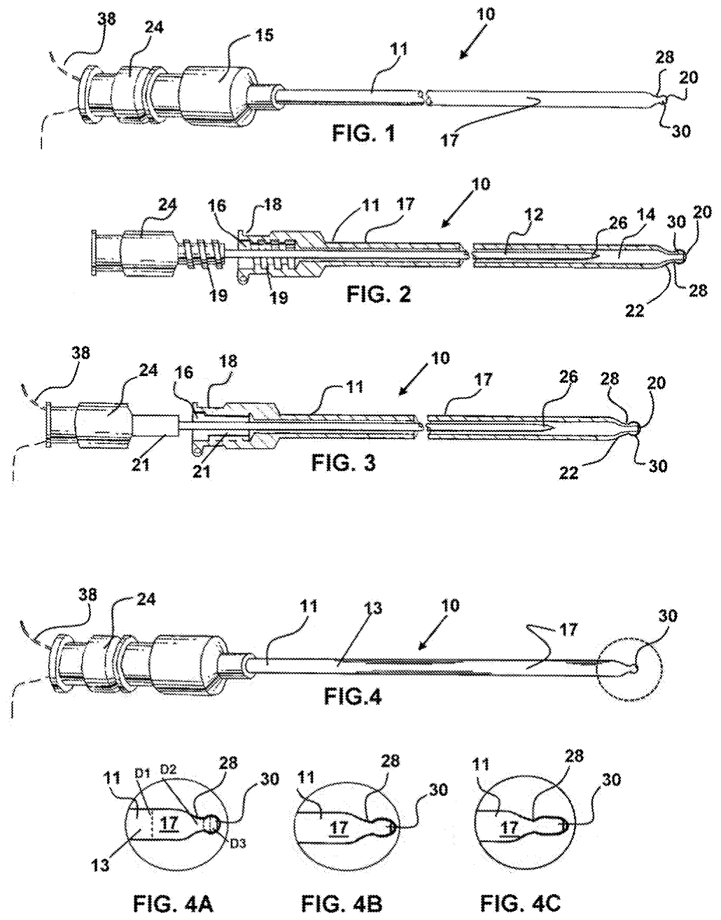

Now referring to drawings in FIGS. 1-8, wherein similar components are identified by like reference numerals, there is seen in FIGS. 1-2, the sheath device 10 herein in an engaged position, wherein the elongated cylindrical body 11 of the sheath or device 10 surrounds a needle 12 extending axially through an axial passage 14 of the cylindrical body 11 of the device 10. The axial passage 14 communicates between a first opening 16 at a first end 18 of the cylindrical body 11 of the sheath device 10, to a second opening 20 located at a distal end 22 thereof.

As shown, a first end 16 of the body of the body 11 of the sheath device 10 is adapted to operatively engage with a handle 24 or similar component which is engaged to a first end of the needle 12 to position the needle totally withing the axial passage 14. Such an engagement may be sliding between the two or for instance cooperative threads on the body 11 and the handle 24 or frictional engagement.

This of the body 11 at the first end 18 thus is adapted to engage the body 11 to the handle 24 of the needle, in a manner whereby manipulation of the handle 24, while holding a grip 15 or other part of the sheath device 10, will translate the needle 12 within the axial passage 14, and thereby translate the distal end 26 of the needle 12, to project through and extend from the second opening 20.

Particularly preferred in all modes of the device 10 herein, is a substantially spherical portion 30 positioned at or adjacent the distal end 22 of the body 11 of the sheath device 10 opposite the first end 18 thereof. Also preferred adjacent to the spherical portion 30 is an annular recess 32 in-between a larger linear first portion of the body 11 of the sheath device 10, and the spherical portion 30 at the distal end 22 of the body 11 defining the sheath device 10.

By substantially spherical is meant any globular or flattened spherical shape, or similar modified spherical shape, which when positioned on the distal end 22 of the body 11 of the sheath device 10, will position a curved exterior surface 17 of the body 11 on the distal end 22 which radially extends from and around the second opening 20 to a communication of the exterior surface 17 with an annular recess 28 in-between the spherical portion 30 and a linear first portion of the body 11 of the sheath device 10. The linear portion extends from a communication with the surface forming the annular recess 28, toward the first end 18 thereof.

Thus the substantially spherical portion 30 can be round like a globe, or for example, be egg-shaped, almond shaped, “blimp” shaped, or otherwise bulbous shaped, having the curved exterior surface of the body extending radially around and away from the second opening 20. The exterior surface 17 shaped to form the bulbosus portion continues to an intersection of the curved exterior surface 17 defining the spherical portion 30, with the exterior surface 17 of the body 11 formed to an annular recess 28. Thus a substantially spherical shape or bulbous curved shape, which positions a curved portion of the exterior surface 17 of the body 11 extending around, and radially from the second opening 20, toward the first end 18 of the body 11, or toward and intersecting and edge of the annular recess 28, is considered within the scope of this patent.

While it is noted that a spherical portion 30 can be placed at the distal end of the body 11 without the annular recess 28, to inclusion of the recess 28 is preferable. The substantially curved shape of the spherical portion 30 positioned immediately adjacent to the adjacent annular recess 28, in addition to significantly reducing lacerations and punctures of tissue as noted above, provides a significantly enhanced locational view on an electronic display. Such is provided on the electronic display when the distal end 22 location of the body 11 of sheath device 10 is seen using ultrasound, RF, or computer aided depictions of the sheath device 10 within the body of a patient.

As noted, shown in FIG. 2 shows a sectional view of the sheath device 10 similar to that of FIG. 1, having a first end 18 of the body 11 forming the sheath device 10, adapted for operative engagement with a handle 24 engaged to a first end of the needle 12. As depicted in FIG. 2 and FIG. 3, this operative engagement allows the user to manipulate the handle 24 relative to the body 11 of the sheath which remains fixed in position, and to translate the distal end of 26 of the needle 12 to extend from the second opening 20, or retract the distal end 26 of the needle 12 entirely back into the axial passage 14.

In FIG. 2, this operative engagement is a threaded engagement between threads 19 on the handle 24 and mating threads 19 on the first end 18 of the body 11 of the sheath device 10. In FIG. 3, this engagement is shown as a frictional or sliding engagement of mating surfaces 21 of the handle 24 with a complimentary mating surface 21 at the first end of the body 11 of the sheath device 10. The depicted modes of operative engagement allow manipulation of the handle 24 relative to the body 11 to axially translate the needle 12 along the axial passage 14 to thereby extend the distal end of the needle, from the second opening 20.

As can be seen in FIG. 4, all modes of the sheath device 10 include the substantially spherical portion 30 is positioned on the distal end 22 of the body 11, adjacent to an annular recess 28. The spherical portion 30 shown in FIG. 4, is encircled by dotted line which refers to the enlarged shapes of substantially spherical portions 20 shown in FIGS. 4A and 4B and 4C. These depictions are exemplars of current preferred configurations of substantially spherical shapes of the spherical portion 30, but which should not be considered limiting as noted herein.

FIG. 4A depicts a spherical portion 30 with a round or more globular spherical shape, which formed on the distal end 22 positions a curved surface which radially communicates around the second opening 20, and toward the annular recess 28. In all modes the annular recess 28 is positioned in-between the spherical portion 30 and the linear first portion 13 of the body 11 of the sheath device 10 extending from the first end 18 to curve of the exterior surface 17 of the body 11 forming one side of the annular recess 28.

Also shown in FIG. 4A are the diameter of the first portion 13 of the body 11 of the sheath device 10, extending from the annular recess 28 toward the first end 18 of the body 11, and the diameter D2 of the annular recess 28, and the diameter D3 of the spherical portion 30. To significantly enhance electronically generated depictions of the sheath device 10 on a video display or the like, it has been found that forming D3 the diameter of the spherical portion 30 to be substantially 20 to 95 percent of the diameter of the first portion D1, and forming the diameter D2 of the annular recess, between 30 to 80 percent of the diameter D3 of the spherical portion 30 works well to yield a more discernable electronic image of the distal end of the body 11 of the sheath device 10. On such electronically generated images from sonic waves or RF energy communicated to the device 10 while within the patient, a dimensioning of the diameter D2 of the annular recess 28 smaller than D3 the diameter of the spherical portion, causes the distal end 22 to be much more clearly visible on the electronic display screen, at a wide variety of angles of the body 11 to the electronic sensor or the sonic generator for ultrasound.

FIG. 4B shows the spherical portion 30 formed with an elongated circular or flattened spherical shape. This shape positions the preferred curved portion of the exterior surface 17 radially extending from and radially around the second opening 20, as in all preferred modes of the device 10 herein. This curved exterior surface 17 of the body 11 extends radially from and around the second opening 20 toward the annular recess 28, which as noted is located in-between a linear first portion 13 of the body 11 and the spherical portion 30 of the body 11 of the sheath device 10.

FIG. 4C depicts the spherical portion 30 formed with a more elongated circular or flattened spherical shape than that of FIG. 4B. This also guards against tissue lacerations and perforations, by positioning the curved portion of the exterior surface 17 radially extending around the second opening 20, as in all preferred modes of the device 10 herein. This curved portion of the exterior surface 17 of the body 11 extends radially from the second opening 20 toward the annular recess 28, located in-between a linear first portion 13 of the body 11 and the spherical portion 30 of the body 11 of the sheath device 10.

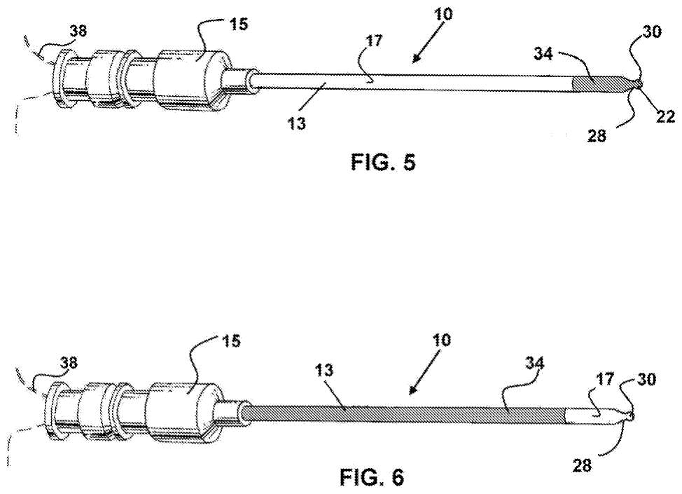

FIG. 5 depicts the exterior surface 17 of the body 11 of the sheath device 10 at and adjacent the distal end 22, configured with an exterior surfacing 34 placed on the circumferential exterior surface 17 of the body 11 of the sheath device 10. As shown in FIG. 8A, this surfacing 34 better reflects RF energy or sonic waves, back to their receiving device to enhance a sonic, RF, or computer-aided video screen depiction thereof.

FIG. 6, depicts the exterior surfacing 34 positioned on the exterior surface 17 of the body 11 of the sheath device 10 along a section of the first portion 13 running from the annular recess 28 toward the first end 18. As with the surfacing 34 of FIG. 5, it better reflects and or focuses or reacts with RF energy or sonic waves from a transducer, to enhance a sonic, RF, or computer aided depiction thereof, which as noted, aids out of plane needle tip visualization as well as angles approaching or extending away from the viewer.

Such surfacing 34 can be formed of reflecting material for RF and sonic waves, from a group of reflective materials, including dimpled or otherwise structural surfacing of the exterior surface 17 of the body 11, or a polymeric coating layer thereon which has nano particles such as nano glass or plastic microspheres encapsulated within the cured polymeric coating layer upon the exterior surface 17 of the body 11 of the device 10.

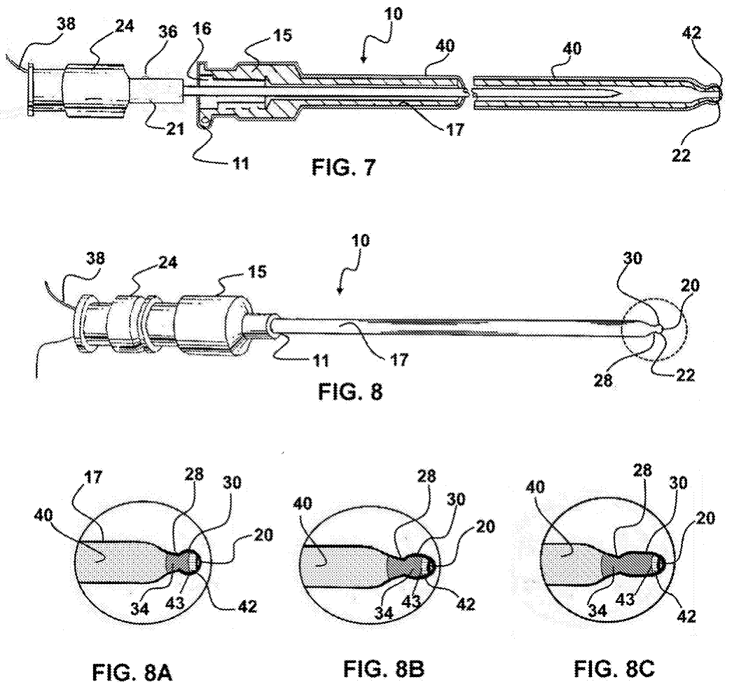

Shown in FIG. 7, is a mode of the device 10 having the body 11 of the sheath formed of material which is electrically conductive and is employable with echogenic stimulating needles which are widely available and can be energized with an electric current by the physician to thereby stimulate nerves of a patient to ascertain if the needle 12 is properly positioned. By forming the sheath body 11 of conductive material such as metal, the electric current can be communicated to the body 11 by a direct wired connection or through an electric connection from the needle 12 which receives current from an electric current supply 38, such as an electric contact 36 on the grip 15.

As shown in FIG. 7, the exterior surface 17 of the body 11 is coated with a layer of an insulating coating 40 covering all the exterior surface 17 but for an exposed portion 42 of the conductive exterior surface 17. This exposed portion 42 shown in FIGS. 7 and 8A-8C, is located preferably adjacent to or formed as a ring encircling the second opening 20 from which the distal end of the needle may project. By positioning this exposed portion 42 immediately adjacent the opening 20, the user can employ a switch or otherwise energize the electric current supply 38 to communicate electric current to electrically energize the body 11 of the sheath. The electric current will be communicated through the conductive body 11 only to body tissue, contacting only the exposed portion 42 adjacent the second opening 20. In this mode the physician does not have to project the needle 12 from the opening 20, which can injure the patient, to ascertain if the opening 20 is in the desired position for a nerve block.

FIG. 8 shows the device 10 as in FIG. 7 with the insulated exterior surface 17 extending to the a exposed portion 42 which is ring shaped. In this ring shape, the exposed portion 42 entirely surrounds the second opening 20, which provides more area of contact for the current to flow to body tissue such as nerves when the electric current is energized. Thus forming the exposed portion 42 as a ring, surrounding the second opening 20 is preferred for more accurate communication of electric current to nerves and tissue immediately adjacent the opening 20 from which the needle will deploy. Also shown in FIG. 8 is an encircling dotted line the tip of the body 11, which is depicted in enlarged modes in FIGS. 8A-8C.

FIG. 8A shows the tip of the device 10 with the spherical portion 20 herein, having the insulating coating 40, enlarged to show the conductive exposed portion 42 adjacent to or preferably formed as a ring, surrounding the second opening 20. Also shown are an optional sonic or RF reflective surfacing 34 immediately adjacent to the conductive exposed portion 42. By immediately adjacent is meant that one side edge of the gap in the insulating coating 40 defining the exposed portion 42 contacts a terminating side edge of the reflective surfacing 34 positioned on the exterior surface 17 or the insulating coating 40 on that surface 17. Thus a line is formed between the side edge of the area of the exposed portion 42 and a side edge of the area of the surfacing 34. When the reflective surfacing 34 is positioned circumferentially on the exterior surface of the body 11, and the exposed portion 42 is formed in a ring, a line 43 is formed between them and encircles the exterior of the body 11 making the distal end 22 highly visible using RF or sonic electronic rendering during use, from any angle.

It has been found beneficial in testing to locate the reflective surface 34 in this adjacent position in contact an edge defining the exposed portion 42, gives the surgeon two highly accurate points of positional reference for the same point on the distal end 22 of the device 10. Because the reflective surface 34 and the exposed portion 42 are next to each other, the surgeon can be assured that either or both will provide highly accurate position determination of the same point on the device 10, when for example viewed electronically with the exposed portion 42 energized to cause a muscle response.

FIG. 8B is shown configured with the exposed portion 42 which is conductive and does not have the insulating coating 40, and will communicate electric current to tissue in contact therewith, when it is immediately adjacent the reflective surfacing 34 portion and having an extended or elongated spherical portion 30 at the tip. As shown, the conductive exposed portion 42 is formed as a ring encircling the second opening 20 and located on the curved exterior surface radially surrounding the second opening 20. FIG. 8C is configured the same as FIG. 8B, but has the spherical portion 30 formed in an elongated fashion having linear or very slightly curved sides, which present a smaller cross section for the distal end 22 of the body 11 which can be advantageous in tight quarters of the body of a patient.

While all of the fundamental characteristics and features of the neurovascular puncture-avoidant sheath invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed:

1. A sheath comprising:
a body having an elongated cylindrical shape, said body formed in a unitary structure having a first end opposite a second end of said body;
an exterior surface of said body extending between said first end and said second end of said body;
an axial passage communicating through said body along a straight line extending from an opening formed in said body on said first end thereof to a second opening formed in said body at said second end thereof, said second opening fixed in an alignment with said first opening;
said first end of said body configured for an operative engagement with a handle having a needle projecting therefrom into said axial passage, wherein in said operative engagement, translation of said handle toward said second opening, translates a distal end of said needle to project from said second opening;
said exterior surface of said body having an annular recess formed therein, said annular recess positioned adjacent said second end of said body;
a substantially spherical portion of said exterior surface of said body surrounding said axial passage and positioned at said second end of said body adjacent said annular recess, said spherical portion positioned in-between said annular recess and said second opening; and
said spherical portion having a curved surface, said curved surface extending radially away from said second opening to an edge of said annular recess, whereby a needle with said handle in operative engagement with said first end of said sheath, is protected from contact with body tissue until said manipulation of said handle translates said distal end of said needle to project from said second opening.

2. The sheath of claim 1 additionally comprising:
said body being electrically conductive;
said body communicating an electric current therethrough when in a connection to an electric current supply;
an insulating coating covering of said exterior surface of said body;
said insulating coating covering all but an exposed portion of said exterior surface of said body, said exposed portion positioned in-between said second opening and said annular recess; and
only said exposed portion of said exterior surface communicating said electric current to body tissue contacting it.

3. The sheath of claim 2, wherein said exposed portion is located upon spherical portion of said body.

4. The sheath of claim 3 wherein said exposed portion is formed in a ring upon said curved surface extending radially away from said second opening, said ring of said exposed surface completely surrounding said second opening.

5. The sheath of claim 1 additionally comprising:
a surfacing positioned on said exterior surface of said spherical portion of said body; and
said surfacing configured to enhance reflections of sound from a transducer employed to produce ultrasound images.

6. The sheath of claim 2 additionally comprising:
a surfacing positioned on said spherical portion of said exterior surface of said body;
said surfacing configured to enhance reflections of sound from a transducer employed to produce ultrasound images; and
a side edge of said surfacing being adjacent to a side edge of said exposed portion.

7. The sheath of claim 3 additionally comprising:
a surfacing positioned on said spherical portion of said exterior surface of said body;
said surfacing configured to enhance reflections of sound from a transducer employed to produce ultrasound images; and
a side edge of said surfacing being adjacent to a side edge of said exposed portion.

8. The sheath of claim 4 additionally comprising:
a surfacing positioned on said spherical portion of said exterior surface of said body;
said surfacing configured to enhance reflections of sound from a transducer employed to produce ultrasound images; and
a side edge of said surfacing being adjacent to a side edge of said exposed portion.

9. The sheath of claim 3 wherein said spherical portion has a diameter which is between 20 to 95 percent of a diameter of a first portion of said body extending between said first end and said annular recess.

10. The sheath of claim 4 wherein said spherical portion has a diameter which is between 20 to 95 percent of a diameter of a first portion of said body extending between said first end and said annular recess.

11. The sheath of claim 5 wherein said spherical portion has a diameter which is between 20 to 95 percent of a diameter of a first portion of said body extending between said first end and said annular recess.

12. The sheath of claim 6 wherein said spherical portion has a diameter which is between 20 to 95 percent of a diameter of a first portion of said body extending between said first end and said annular recess.

13. The sheath of claim 7 wherein said spherical portion has a diameter which is between 20 to 95 percent of a diameter of a first portion of said body extending between said first end and said annular recess.

14. The sheath of claim 8 wherein said spherical portion has a diameter which is between 20 to 95 percent of a diameter of a first portion of said body extending between said first end and said annular recess.
